(19) United States
(12) Patent Application Publication
(10) Pub. No.: US 2024/0095218 A1
(43) Pub. Date: Mar. 21, 2024

(54) CUSTOMIZABLE DELIMINATED TEXT COMPRESSION FRAMEWORK

(71) Applicant: KONINKLIJKE PHILIPS N.V., EINDHOVEN (NL)

(72) Inventor: Yee Him CHEUNG, Cambridge, MA (US)

(21) Appl. No.: 17/768,878

(22) PCT Filed: Oct. 15, 2020
(86) PCT No.: PCT/EP2020/078996

§ 371 Date: Apr. 14, 2022

Related U.S. Application Data
(60) Provisional application No. 62/923,113, filed on Oct. 18, 2019.
(60) Provisional application No. 62/956,941, filed on Jan. 3, 2020.

(51) Int. Cl. G06F 16/174; G06F 16/17

(52) U.S. Cl. CPC G06F 16/1744; G06F 16/173

(57) ABSTRACT

A method for compressing data includes obtaining a compression schema customized to a format of a delimited text file, and using the compression schema to parse the delimited text file into a plurality of data blocks, split each of the data blocks into a plurality of data units for efficient selective access, and compress the plurality of data units in the plurality of data blocks using different compression algorithms for improved compression ratio. The delimited file is split into a plurality of data blocks based on the region definitions in the schema. Each of the plurality of data blocks is split into the plurality of data units based on its respective data unit size specified in the schema. The plurality of data units in each of the plurality of data blocks are compressed using the different compression algorithms indicated by the compression instructions in the schema. The compressed file consists of the compressed data blocks, the compression schema and various metadata for data decompression, file reconstruction and functionalities such as data security and search query. The delimited text file may include genomic information or another type of information.

### CROSS-REFERENCE TO RELATED APPLICATIONS

This application is related to U.S. Provisional Patent Application Ser. No. 62/923,113, filed on Oct. 18, 2019, the entire contents of which are hereby incorporated herein by reference for all purposes.

This application is related to U.S. Provisional Patent Application Ser. No. 62/923,141, filed on Oct. 18, 2019, the entire contents of which are hereby incorporated herein by reference for all purposes.

This application is related to U.S. Provisional Patent Application Ser. No. 62/956,952 (Attorney Docket No. 2019P00842US01), entitled “System and Method for Effective Compression, Representation and Decompression of Diverse Tabulated Data,” filed concurrently with the present application, the entire contents of which are hereby incorporated by reference herein for all purposes.

### TECHNICAL FIELD

Various embodiments described herein relate to data compression, and more particularly, but not exclusively, to compression of delimited text.

### BACKGROUND

Many large data files, especially in the fields of genomics, bioinformatics and healthcare analytics, are by nature delimited texts, which differ by their row and column definitions and other formatting details. Examples of genomic data in delimited text include variant call files (VCF), gene expression data, browser extensible data (BED), BigBed, GFF3, GTF, Wig, BedGraph, and BigWig, as well as others.

Various techniques have been proposed to compress data and other types of delimited files. One example compression technique is gzip. However, delimited files are not suitable for compression by all types of compression techniques. Also, existing approaches to compressing delimited files use the same algorithm to compress all portions of the file. Also, some compressors lack support for desirable functionalities (such as fast query and random access, encryption, authentication, and access control). For at least these reasons, existing compression performance for delimited files have proven to be suboptimal.

### SUMMARY

A brief summary of various example embodiments is presented below. Some simplifications and omissions may be made in the following summary, which is intended to highlight and introduce some aspects of the various example embodiments, but not to limit the scope of the invention. Detailed descriptions of example embodiments adequate to allow those of ordinary skill in the art to make and use the inventive concepts will follow in later sections.

In accordance with one or more embodiments, a method for compressing data, comprising obtaining a compression schema customized to a format of a delimited text file; parsing the delimited text file into a plurality of data blocks based on the compression schema; splitting each of the data blocks into a plurality of data units based on the compression schema; and compressing the plurality of data units in the plurality of data blocks using different compression algorithms, wherein the delimited text file is parsed into the plurality of data blocks based on the region definitions in the schema; each of the plurality of data blocks is split into the plurality of data units based on its respective data unit size in the schema; and the plurality of data units in each of the plurality of data blocks are compressed using the different compression algorithms indicated by the compression instructions in the schema.

Obtaining the compression schema may include creating a new compression schema or determining the best-matching one from a plurality of compression schemas based on information input by a user or the extension of the delimited text file, wherein each of the plurality of compression schemas customized for respective one of a plurality of different formats of delimited text files.

Obtaining the compression schema may include automatically analyzing or detecting the format of the delimited text file; and automatically generating a new compression schema for optimum compression performance or selecting the best-matching one from a plurality of compression schemas stored in a schema repository, wherein each of the plurality of compression schemas is customized for respective one of a plurality of different formats of delimited text files. Files corresponding to the compression schemas stored in the schema repository have predetermined file extension indicative of the plurality of different formats of the delimited texts files.

The method may include creating the compression schema customized to the format of the delimited text file based on a tool with a graphical user interface, the graphical user interface including predetermined windows to allow for input of information that customizes the compression schema to the format of the delimited text file.

The method may include generating a compressed file consisting of the plurality of compressed data units in the plurality of data blocks, and a compression schema that includes instructions for decompression of the plurality of compressed data units and file reconstruction of the compressed file. The compressed file includes metadata information for decompression, file reconstruction, and extended functionalities. The extended functionalities include data security and search query.

The compressed file may include code and usage definitions of specialized compression/decompression algorithms for portability and accessibility of the compressed file. The compression instructions may indicate the different compression algorithms and their corresponding parameters to be used to compress different ones of the plurality of units based on different content of the blocks.

The compression instructions may indicate a first type of compression algorithm is to be used to compress a first data unit including a first one of the group consisting of a type of values, a type of information, a type of data format, and a type of data arrangement, and a second type of compression algorithm is to be used to compress a second data unit including a second one of the group consisting of a type of values, a type of information, a type of data format, and a type of data arrangement, wherein the first one of the group is different from the second one of the group.

In accordance with one or more embodiments, a method for selective data access comprises receiving information indicative of a region of interest in the data (e.g. range of rows and columns in a table), the region of interest corresponding to one or more data units included in at least one data block in the compressed file; selectively decompressing the one or more data units of at least one data block associated with the region of interest in the compressed file without decompressing other data units in the at least one data block or other data blocks in the compressed file, the one or more data units selectively decompressed based on one or more decompression algorithms indicated by the compression instructions in the compression schema; reconstructing the region of interest from the selectively decompressed one or more data units, the region of interest reconstructed based on the region definitions in the compression schema or any user-defined output format; and outputting information indicative of the reconstructed region of interest.

Determining the compression schema may include determining the compression schema from a plurality of compression schemas, wherein each of the plurality of compression schemas is customized to include decompression information for respective one of a plurality of different formats corresponding to the compressed file. Determining the compression schema may include selecting the compression schema from the plurality of compression schemas stored in a schema repository.

The method may include selectively accessing the one or more data units based on a query of the compressed file, the query performed based on one or more terms or range of values found in one or more data units that are selectively decompressed. The delimited text file may include genomic information and wherein the region of interest can correspond to a selected range of genomic coordinates or gene IDs.

In accordance with one or more embodiments, a system for compressing data comprises a schema manager configured to allow users to create, select or auto-generate a compression schema customized to a format of a delimited text file; a parser configured to parse the delimited text file into a plurality of blocks based on the region definitions in the compression schema; a splitter configured to split each of the blocks into a plurality of data units based on its respective data unit size specified in the compression schema; and compression manager configured to compress the plurality of data units in the plurality of data blocks using different compression algorithms indicated by the compression instructions in the compression schema.

The schema manager may create a new compression schema or determine the best-matching one from a plurality of compression schemas based on information input by a user or the extension of the delimited text file, wherein each of the plurality of compression schemas customized for respective one of a plurality of different formats of delimited text files. The schema manager may automatically analyze or detect the format of the delimited text file, and automatically generate a new compression schema for optimum compression performance or select the best-matching one from a plurality of compression schemas stored in a schema repository, wherein each of the plurality of compression schemas is customized for respective one of a plurality of different formats of delimited text files.

The compression manager may extract the codes of the compression algorithms from the compressor repository or metadata of specialized compressors, instantiate the compressors for each data block by allocating computational resources and memory, and running and monitoring the compression of the data units.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate example embodiments of concepts found in the claims and explain various principles and advantages of those embodiments.

These and other more detailed and specific features are more fully disclosed in the following specification, reference being had to the accompanying drawings, in which:

FIG. 1 illustrates an embodiment of a method for generating a compression scheme for a delimited text file;

FIGS. 2A and 2B illustrate example(s) of an instruction table for a first compression schema;

FIGS. 3A and 3B illustrate example(s) of an instruction table for a second compression schema;

FIG. 4 illustrates an embodiment of a system for deconstructing and compressing a delimited text file;

FIG. 5 illustrates an embodiment of a method for deconstructing and compressing a delimited text file;

FIG. 6 illustrates an embodiment of a system for decompressing and constructing from a compressed delimited text file;

FIG. 7 illustrates an embodiment of a method for decompressing and constructing from a compressed delimited text file;

FIG. 8 illustrates an embodiment of a method for selecting and decompressing one or more blocks in a compressed delimited text file that correspond to a region of interest; and

FIG. 9 illustrates an embodiment of a processing system that may be used to implement the operations of the embodiments described herein.

### DETAILED DESCRIPTION

The description and drawings presented herein illustrate various principles. It will be appreciated that those skilled in the art will be able to devise various arrangements that, although not explicitly described or shown herein, embody these principles and are included within the scope of this disclosure. As used herein, the term, “or,” as used herein, refers to a non-exclusive or (i.e., and/or), unless otherwise indicated (e.g., “or else” or “or in the alternative”). Additionally, the various embodiments described herein are not necessarily mutually exclusive and may be combined to produce additional embodiments that incorporate the principles described herein.

One or more embodiments described herein relate to a system and method that provides a data representation and compression framework for various types information, including but not limited to genomic and/or bioinformatics data. In one application, the system and method provide a data representation and compression framework for delimited text files. Unlike other methods which have been proposed, different portions of the same delimited text file may be parsed and compressed using different compression techniques. The compression techniques used for each portion may be optimized for compression of the data in that portion, which may not be optimal for other portions. Thus, a delimited text file may be compressed in a customizable and optimizable manner for the specific portions of the same file or specific types of files under consideration. Also, in at least some embodiments, the file data may be represented and compressed using advanced functionalities that facilitate downstream data screening, manipulation, and analysis.

Moreover, compressing different portions of the same delimited text file separately (using either the same or different compression algorithms) may allow only selected portions of the delimited text file to be retrieved, decompressed, and constructed, independent from other portions of the same file which are not of interest. This improves the efficiency of decompression and allows for access to only those portion(s) of the file independent from other portions. Accordingly, various embodiments present a customizable delimited text compression (CDTC) framework that may be easily and flexibly tailored for the lossless compression of diverse data formats in delimited text for efficient storage and processing.

Compression Schema

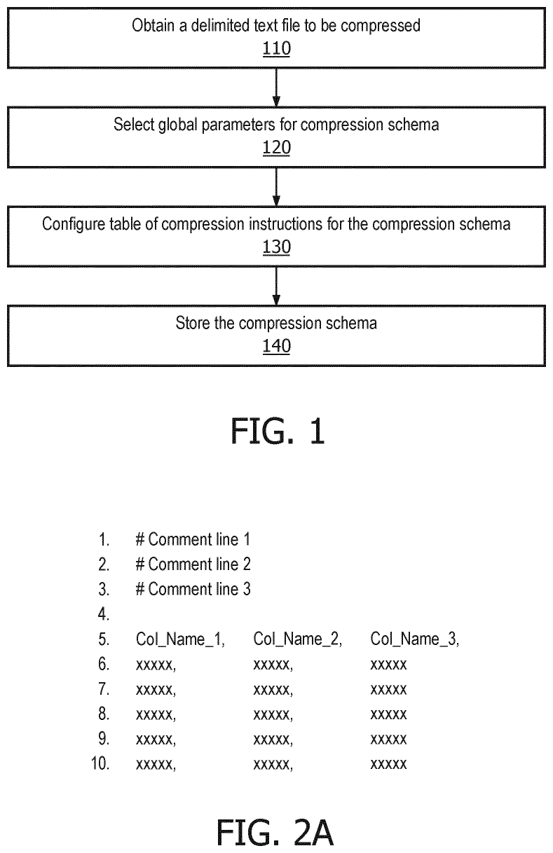

FIG. 1 illustrates an embodiment of a method for generating a compression schema, which, for example, may be used to deconstruct and compress different portions of a delimited text file using different compression algorithms and which may also be used as a basis for selectively decompressing and constructing portions of the delimited text file that has been compressed. Based on the compression schema, users may easily and flexibly customize how a delimited text file is to be divided into different components (e.g., partial lines, lines, rows, columns, matrices, etc.), and how each component may be compressed (e.g., by a different one of a plurality of compression algorithms and their corresponding parameters) and stored. In one embodiment, the compression schema may include a list of global parameters and compression instructions arranged in a predefined format, including but not limited to a table format.

Referring to FIG. 1, the method includes, at 110, obtaining a delimited text file to be compressed and subsequently decompressed. The text file may have any size and may include any type of data, but at least one embodiment may be especially suitable for storing large size files. For example, in one particularly useful application, the text file may include genomic information that is to be deconstructed into data blocks and individually compressed into data units for subsequent storage and uses for research or other purposes.

The text file may be delimited in the sense that it is in a format where each line represents a unit or block and has fields that are separated by a delimiter symbol or value. In another embodiment, a unit or block may correspond to another size or portion of the file, such as a portion of a line, a predetermined group of lines, or one or more other types, sizes, or sections of the text file respectively separated (or delimited) from one another by predetermined symbol(s) or value(s). The units or blocks into which the file is separated may have the same size or at least a portion of them may have different sizes, for example, according to the manner in which the schema is to be defined.

At 120, a set of global parameters are selected that define a compression schema, for example, given the specific type of information contained in the delimited text file. The parameters may define the delimiters, default data unit sizes and default generic compression algorithms to be used on different portions of the file, among other information. In accordance with one embodiment, the following set of global parameters may be selected and defined for the compression schema.

Input File. The schema parameters may include a pointer to the delimited text file to be compressed. The pointer may, for example, indicate the location(s)/address(es) of a memory or other storage device where the delimited text file is stored in uncompressed form. The memory may be remotely located from a processing system implementing the embodiments described herein or may be locally coupled to the processing system. In one embodiment, the memory or other storage device may be connected to the processing system through one or more networks, including but not limited to virtual private networks, the internet, a cloud-based network, or another type of network.

Delimiters. The schema parameters may also include one or more symbols that serve as delimiters in the text file. These symbols may separate the data and other information in the text file into individual fields or components of the same nature that can be collectively compressed due to their common data characteristics. The fields or components may correspond to any of the fields or components described herein. In one embodiment, a row in the text file may be separated by one or more symbols (delimiters) in a way that splits each row (e.g., unit) into one or more columns in the file. An example of a delimiter symbol is the tab symbol (′ \ t′).

In one embodiment, as will be described in greater detail below, the file may include one or more columns of data, which, for example, may be referred to as a data block. Each data block may include one or more data units; that is, in some cases the entire data block may be considered to be a single data unit and in other cases the data block (e.g., column of data) may include a plurality of data units.

Encap Symbol. The schema parameters may also include encapsulation symbols that indicate that text in between the symbols should not be split into columns by delimiters, if any. An example of an encapsulation symbol is the double quote symbol (”).

Comment Symbol. The schema parameters may also include a comment symbol that marks a comment line at the beginning of a portion of the text file, e.g., at the beginning of a row. Comment lines may remain intact and be stored together in a file part, with a default block name (e.g., “Comments”) after the delimited text file has been deconstructed. This may include comment lines in regions defined in the compression instructions. An example of a comment symbol is the hash character (′#′).

Gen Comp Alg. The schema parameters may also indicate a general compression algorithm to be applied on blocks for which no specific compression algorithm has been designated in the schema. As described herein, in one or more embodiments different data blocks, each consisting of one or more data units, of the delimited text file may be compressed using different compression algorithms. In the case where a compression algorithm has not been indicated in the schema for a particular data block, that data block may be compressed by the general compression algorithm specified by this parameter. Thus, the general compression algorithm may be considered to be a default algorithm when no other algorithm has been specified.

In one embodiment, the entire file, i.e. all data blocks and their respective data units, may be compressed using the same or different compression algorithms. In another embodiment different portions of the file may be selectively compressed using, for example, different compression algorithms. For example, the file may include one or more columns of data, which, for example, may be referred to as a data block. Each data block may include one or more data units; that is, in some cases the entire data block may be considered to be a single data unit and in other cases the data block (e.g., column of data) may include a plurality of data units. In a selective-compression embodiment, for example, compression is only applied on selected data blocks or data units using their respective algorithms as described in the schema, while the rest is stored without compression. This approach is useful when certain data blocks are frequently accessed or queried, and should therefore remain uncompressed for ease of data retrieval.

Default Data Unit Size. The schema parameters may also indicate a default number of rows that form or define a data unit for compression. In one embodiment, this parameter may indicate a predetermined fixed integer value. In one embodiment, this parameter may indicate that a processor should execute an algorithm that implements an “Auto” function, which involves automatically selecting the size for each block based on the impact on compression ratio and decompression speed of a single data unit for selective access. In one embodiment, the parameter may indicate an “Inf” function should be performed, which involves compressing the data block as a whole without splitting the data block into individual data units.

Output Folder. The schema parameters may also include output folder for storing the compressed data parts and associated metadata. Examples of the metadata are discussed in greater detail below.

At 130, a table of compression instructions may be generated/customized and included in the schema. When a table of compression instructions is included, each row may define (i) a specific region in the delimited file for data extraction and (ii) how the extracted data should be represented and compressed. This table may indicate that different compression algorithms are to be used to compress different ones of the specific regions.

Thus, such a table may include information for instructing a processor to compress different regions (or portions) of the data file using different compression algorithms. This may be beneficial for a number of reasons. For example, the data or information in one region or portion of the file may be compressed by one algorithm that has been determined to be more efficient for that type of data or information. The data or information in other regions or portions of the file may be compressed by another algorithm that is more efficient for the data or information in those portions.

In one embodiment, the table of compression may be configured to include fields designating the types of information indicated below.

Region Lines. The table may include a field indicating a range of line numbers of a rectangular region (or other unit or block) in the delimited text file on which a current row of compression instructions should be applied. For example:
“100:500” may define a region from line 100 to line 500 inclusive
“100:” may indicate a region that starts from line 100 and continues until a blank line or end of file is reached.

If the table does not specify the range of lines for a row, then control software may instruct the system processor to use the same range of lines as was used in a previous row. And, if the row is a first row, then the control software may instruct the system processor to start from an upcoming non-comment/empty line until it hits a blank line or end of file.

Region Cols. The table may include a field indicating a set of column indices of the rectangular region (or other unit or block) in the delimited text file on which a current compression instruction should be applied. This may be, for example, as follows:
“11:15” may indicate extracting columns 11 to 15 into a matrix with five columns
“11:2:15” may indicate extracting columns 11, 13 and 15 (at intervals of 2) into a matrix with three columns
“11-15” may indicate collapsing columns 11 to 15 into one column with delimiters retained

If not specified, the rest of the lines (after the rightmost column defined previously for the same range of lines) may be extracted as one column and not further split by delimiters.

Data Type. The table may also include a field indicating a type of data element. Examples of these types include string, fstring (formatted string), char, int, uint (unsigned integer), float, etc. The number of characters or bits may be specified, for example, in brackets, e.g. char(8) means 8 characters and uint(8) means eight-bit unsigned integer. For the fstring data type, the string format may be specified in a bracketed string, e.g. fstring(′rs % uint(24)′) represents string elements that begin with the prefix “rs” followed by an unsigned integer. If not specified, the data type may be automatically selected by the system processor to correspond to a default type or to optimize performance. In addition, a “key” qualifier can be included in the data type definition if the values in the data block will be used for query access. In such cases, a search index will be generated for the data block and stored separately as a metadata component.

Comp Alg. The table may also include a field indicating the names of the compression algorithms and their parameters, if any, for respective ones of the regions/blocks in the delimited text file. In one embodiment, the type of compression algorithm to be used may be determined based on the content of the region/block to be compressed. For example, a region/block including numerical values may be compressed using an algorithm different from the algorithm for formatted strings. In some embodiments, if there exists multiple data elements in a formatted string, then comma-separated compression algorithms may be specified for each of the data elements in the same order. The following is a non-exhaustive list of examples of compression algorithms that may be indicated:
“RLE” (Run Length Encoding): This type of compression algorithm may be used for compressing long consecutive data elements of the same value, e.g. genotype values of single nucleotide polymorphism (SNP) array data.
“Delta” (Delta Encoding): This type of compression algorithm may be applicable to numerical values, coding only the difference between the current and previous elements, rather than storing the whole value. This algorithm may be used, for example, on genomic coordinates.
“Enum” (Enumeration). This type of compression algorithm may be used if the data to be compressed includes repeated items selected from a small set of possible values. In this case, compression may be achieved by coding each unique value with a fixed, minimum number of bits long enough to cover all possible values. Enumeration compression may be used, for example, on functional annotation of variants (missense, non-sense, silent, frameshift, splice-site, etc.).
“Index”. This type of compression algorithm may be used if the data to be compressed includes a series of values with a fixed format and a numeric component that increases or decreases at regular intervals. In this case, compression may be performed by deriving and storing: (i) the data format, (ii) the initial value of the numeric component, (iii) the interval, and (iv) number of elements.
“Sparse”. This type of compression algorithm may be used if the data to be compressed includes a sparse matrix with most elements in default value. In this case, compression may be performed by transforming the matrix into a Matrix Market-like coordinate format that only contains the row index, column index and values of non-default entries. Furthermore, any symmetry property of the matrix may be exploited by storing only entries from the lower triangular portion. This approach may be used, for example, on the genotype values of NGS data.
General Purpose Compressor. Users can specify one of the general purpose compression algorithms (e.g., gzip, bzip2, 7-zip or arithmetic coding) for compressing general data types that do not fall into any of the prior categories.
Combined Algorithms. Users can specify a concatenation of coding algorithms to be performed sequentially on a data unit. For example, “Enum+RLE” means to first transform the original data into enumeration code and then apply RLE on the transformed values.
“Auto”. The value indicates to let the software controlling the system processor select the compression algorithm based on an analysis of the data. The selected compression algorithm should be noted for proper decompression.
“Default” or “ ” (blank). The value indicates that the general compression algorithm defined in the global parameter Gen_Comp_Alg should be applied.
“Original”. The value indicates that the original data in a region/block of the delimited text field should be saved without compression. This may allow for faster selective access queries on the data fields.

Data Unit Size—The table may also include a field indicating whether the data unit size deviates from the default value in the global parameter Default_Data_Unit_Size. Similarly, its value could be an integer, “Auto” or “Inf”.

Column Name. The table may also include a field indicating the name(s) of the column(s) covered by the defined region. In one embodiment, a user may specify a comma-separated string of column names or use the reserved expression “First_Row” to indicate that the first row contains the column name(s) and should not be compressed with the rest of the rows. If not specified, a name may be auto-generated for each column.

Block Name. The table may also include a field indicating a name that uniquely identifies the data compression block. If not specified, Column_Name may be used.

In one embodiment, a user may create a compression and associated decompression algorithm in order to process special data types. To protect against malicious software, each compressor/decompressor may be accompanied by a digital signature as a proof of origin and authenticity. In some embodiments, such a digital signature may be required for user-created algorithms. The executables, together with their digital signatures, may be imported to the compressor/decompressor repository along with their associated IDs and method signature (list of input parameters) to be used in schema definitions or stored as part of the compressed data file for portability and accessibility. In some scenarios, an algorithm may require data from another column or block as inputs. This may be supported, for example, by users specifying the column/block name prefixed by a special character such as “8” as part of the method signature in Comp_Alg.

The rows in the instruction table may be ordered based on the locations of the defined regions. In one embodiment, the region with smaller beginning line numbers should come first. If the beginning line numbers of multiple regions are the same, then the region with the smaller beginning column index may come first. Also, blocks of whole lines not covered in the instruction table may be aggregated together with other comment/blank lines for compression. Their line numbers in the original text may be stored as metadata for future file reconstruction. Any other regions missing from the instruction table may be identified by the software as individual blocks to be compressed using the algorithm defined in the global parameter Gen_Comp_Alg. In some embodiments, a Region_Error may be returned if there are any ambiguities or overlaps in the region definitions. In one embodiment, the definitions of global parameters and instruction tables may be interspersed in the schema, in order to allow the global parameters to be changed in between the compression instructions.

The instructions for a group of blocks may be marked by labels such as <Blocks></Blocks>, and an individual block may be marked by a label such as <Block></Block>. The fields described above may then be specified as attributes to these labels. In at least one implementation, each block may be split into sub-blocks, for example, through a nested block structure.

In some embodiments, the beginning and end of each data table may be enclosed by labels such as <Table></Table>. The following are some examples of attributes that may be applied:
ID—Name of the table
Start Line—the line number of the first row in the table, including the header, if it exists. If not specified, start from the current position of the file parser.
Num Rows—the number of rows in the table. If not specified, the table may end when it hits a blank line or end of file.
First Row Col Names—If true, the first row contains column names to be processed separately from the data entries and stored in the metadata. The default value may be false.
First Col Row Names—If true, the first column contains the row names to be processed separately from the data entries and stored in the metadata. The default value may be false.
Col Names—List of column names in the same order as the columns in the table.
Row Names—List of row names in the same order as the rows in the table.
Col Span—List of integer values, each corresponding to a column name and indicating the number of data columns associated with the column name. This may be useful for grouping multiple data columns under the same header. If not specified, there may be assumed a one-to-one mapping between the column names and the data columns.

In the table definition, the same data element (e.g., column name) may be defined at the table or block levels. In such cases, the later value may override the former one. Data elements in a table may be referred to following a hierarchical naming approach. For example, one table may have an ID “Tab1” with four columns, where the first two columns are named “Col_1” and “Col_2” and columns 3 and 4 are grouped under the name “Cols_3_4”. Then, all columns may be referred to as Tab1.cols, the first column as Talatcol[1] or Tabtcol[“Col_1″], and the fourth column as Tabtcol[4] or TabEcorCols_3_4”][2] (e.g., the second column grouped under “Cols_3_4”).

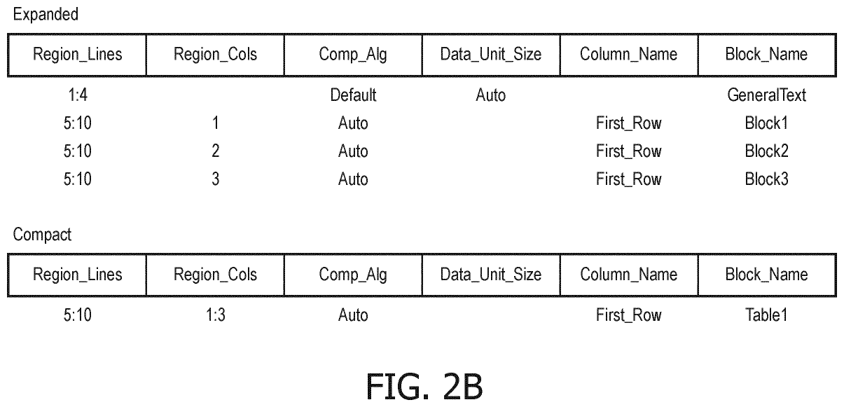

FIGS. 2A and 2B illustrate an example(s) of an instruction table for a first type of compression schema that illustrates how blocks for compression may be defined. In FIG. 2A, information is included for partitioning original delimited text into blocks to be individually compressed. In FIG. 2B, associated instruction tables are illustrated for performing the partitioning in expanded and compact forms, which are equivalent. Since the compact table refers to a region starting from the fifth line, the first four rows in the file should be compressed as general text. Rows 2-4 in the expanded form may be collapsed into a single row in the compact form, since the same compression instruction applies to the three columns. The “First_Row” entries indicate that the column names should be extracted from the first row of the respective columns.

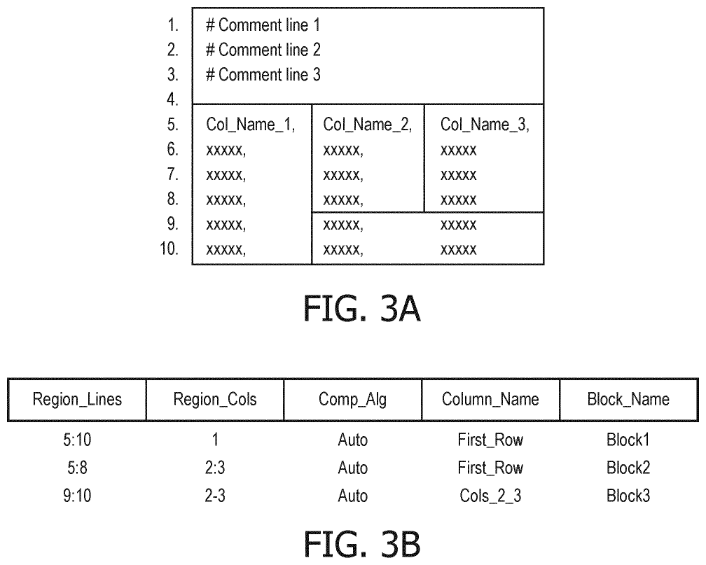

FIGS. 3A and 3B illustrate an example of an instruction table for a second type of compression schema that illustrates how blocks for compression may be defined. In FIG. 3A, information is included for partitioning original delimited text into blocks to be individually compressed. In FIG. 3B, an instruction table is illustrated for performing the partitioning. Since the table refers to a region beginning from the fifth line, the first four rows should be compressed as general text. For lines 5 to 8, the colon in “2:3” indicates that columns 2 and 3 should be separately compressed and stored. Whereas for lines 9 to 10, the hyphen in “2-3” indicates that columns 2 and 3 should be merged into a single column for compression.

The use of a compression schema is especially beneficial for at least some applications, as a user may design the compression schema according to the particular application. This schema and its attendant compression and decompression features, therefore, allows one or more of the embodiments to be customized, while at the same time allowing for selective access of only those portions (e.g., data blocks, data units in a data block, etc.) to be decompressed without having to decompress other portions of the compressed file. This not only allows only specific portions of a compressed file to be targeted for access, but also precludes other portions (e.g., that are not immediate interest) from being decompressed, thereby speeding up the process of accessing targeted portion of genomic data, when the file is directed to such an application.

At 140, the compression schema is stored in a storage area, such as but not limited to a schema repository. The compression schema may be subsequently retrieved to guide a processor (e.g., implementing various managers and other logic) to perform operations including deconstructing a delimited text file, compressing different portions of the deconstructed file using different compression algorithms, decompressing the compressed portions of the file, and reconstructing the file from the decompressed portions. The compression schema may include or be stored in association with metadata as described herein.

File Deconstruction and Compression

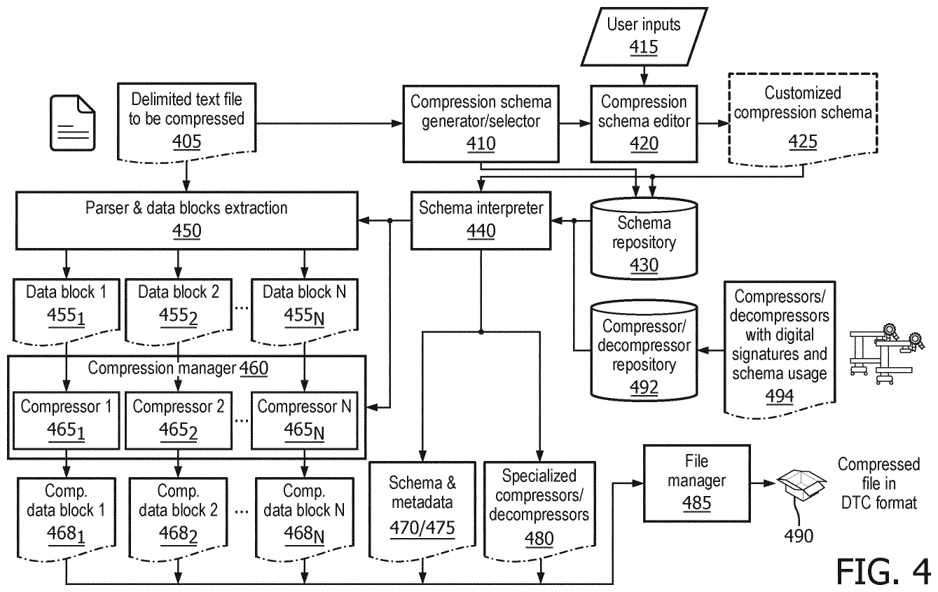

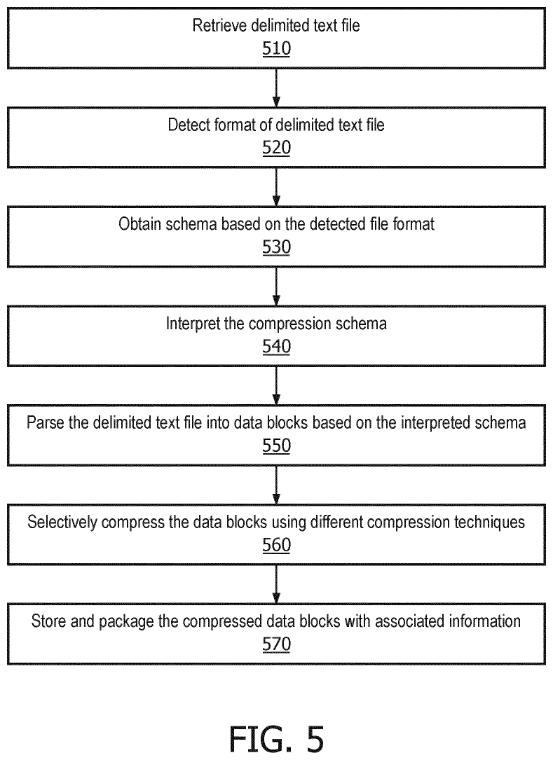

FIG. 4 illustrates an embodiment of a system for deconstructing and compressing a delimited text file, which, for example, may include genomic information. FIG. 5 illustrates an embodiment of a method for deconstructing and compressing a delimited text file, which, for example, may be performed by the system of FIG. 4

Referring to FIGS. 4 and 5, the method includes, at 510, uploading a delimited text file 405 from a data source to a file manager of the system. The data source may be, for example, a computer or other type of processing system which capture and/or stored the data as originally obtained. For example, when the data corresponds to genomic information, the data may be originally obtained from laboratory equipment. The data may have been uploaded directly from the laboratory equipment or may have been stored in raw or pre-processed form. In one embodiment, the data is pre-processed to conform to the data representation formatted and arranged in accordance with the embodiments described herein. When represented, formatted, or otherwise structured in this manner, compression of different blocks (and/or different data units within one or more of the data blocks) of the delimited text file may be performed in an efficient manner.

At 520, the data format of the delimited text file is detected. This may be accomplished, for example, by detecting a file extension of the delimited text file. The file extension or other information indicative of the file format may be detected, for example, by a compression schema generator or selector or by other managing logic.

At 530, a compression schema is determined or selected that corresponds to the format of the delimited text file that was detected. This operation may be performed, for example, by a compression schema generator/selector 410, either alone or in combination with one or more other features. For example, if there exists a pre-defined schema associated with the file extension of the delimited text file, then the compression schema generator may retrieve the schema from a schema repository 430, which was previously loaded and stored with the schema for use with delimited text files having a corresponding compatible format.

If the format of the delimited text file is a new file format, a user may define and import a compression schema for the new file format. For example, this may be accomplished by a compression schema editor 420, which receives and generates a customized compression schema 425 for the new file format based on user inputs 415. In one embodiment, the compression schema editor 420 may be a compression schema creation tool which assists a user in defining the new schema with supporting functionalities, which, for example, may include (i) auto-generation of compression schema through analysis of the delimited text and (ii) user interface for schema customization with auto-suggestions for compression methods and parameters. The customized compression schema may then be stored in the schema repository in association with one or multiple file extensions for future use.

In one embodiment, format of the delimited text file and/or the compressed format generated by the compression schema may include embedded codes (e.g., a compressor executable within the file format itself) with appropriate security protections. The code may be used, by the same or a different entity, to decompress at least selected portions of the compressed file corresponding to the embedded code. The embedded code may be included irrespective of the compressor or content of compressed data, but may be especially beneficial for content compressed using a customized compression algorithm. The code may also be used to compress data as needed.

At 540, a schema interpreter 440 interprets the compression schema determined to correspond to the detected format of the delimited text file. The schema may be interpreted in various ways. For example, interpretation of the compression schema may include updating global parameters in runtime memory with values defined in the schema. These new values may only be used in subsequent instructions. In some embodiments, a compression instruction may only be active when parsing of the delimited text (e.g., line-by-line from top to bottom, and for each line, column-by-column from left to right) has entered a rectangular region associated with the instruction. For each active instruction, a buffer may be created to hold the vector or matrix of values extracted from the associated region, and a compressor may be set up according to the defined algorithm(s) and parameter(s).

At 550, the delimited text file is parsed to extract a plurality of blocks 4551 to 455N in conformance with the schema interpreted by the schema interpreter. The blocks may be split into data units of the same size or at least a portion of them of different sizes. The different sizes may be determined randomly or in accordance with the corresponding schema. The parsing operation may be performed by parser and data extraction logic 450 in a variety of ways. For example, the delimited text file may be parsed line-by-line to generate a corresponding plurality of blocks. This may be performed, for example, by splitting each line of the delimited text file into tokens using delimiters and then assigning each token to a block buffer according to its line number and column index. The tokens in each buffer may then aggregated into data units of pre-defined sizes for compression. In another embodiment, the delimited text file may be parsed into two-dimensional blocks. Once the blocks are generated, they are input into a compression manager.

At 560, the compression manager 460 compresses the blocks using one compression technique or multiple compression techniques. For example, the compression manager may include a plurality of compressors 4651 to 465N, where N 1. Each of the compressors 4651 to 465N may implement a different compression algorithm to compress one or more of the blocks generated by the block extraction logic. The compressor/algorithm to be used to compress each block is determined based on information corresponding to the interpretation of the applicable schema output from the schema interpreter. In one embodiment, compression of the blocks by the different compressors may be performed in parallel to achieve improved efficiency and performance. While FIG. 4 illustrates that the parsed blocks are in one-to-one correspondence with the compressors, in one embodiment any one or more of the compressors may compress a plurality of blocks.

At 570, the compressed blocks 4681, 4682, . . . 468N are stored in respective storage areas of an archive. In one embodiment, the compressed blocks may be stored as individual file parts, along with a master index table that identifies the location of each compressed block for supporting random data access. One or more storage devices may include the storage areas. For example, the storage devices may be one or more buffers, database locations, memories, caches, or other types of data storage.

Various types of information may be stored with or in association with the compressed blocks. The information may include, for example, the compression schema 470 used to parse the delimited text file and/or metadata 475 describing or otherwise linked to respective ones of the blocks that have been compressed. Examples of metadata include row and column names of a table, specific compression algorithm auto-selected (not specified in the schema) for a data block, and delimiter symbol (when more than one delimiter symbol is used) for each block. To facilitate fast random access to specific lines and columns or query by specific terms, the metadata may also include indexing information. The executables of any specialized compression and decompression algorithms 480 required for any data blocks, together with their IDs and method signature, may also be stored to improve the portability and accessibility of the compressed file.

Additionally, or alternatively, information identifying the specific types of compression algorithms used by the compressors to compress respective ones of the blocks may be stored with corresponding ones of the blocks, or in a table linking the types of compression algorithms used for each of the compressed blocks.

At 570, all the generated file components, including the compressed blocks, schema, metadata, and any specialized compressors and decompressors, may be organized and packaged into an archive 490 through a file manager 485.

In another embodiment, rather than storing the compressed data units, schema and metadata as file parts in an archive, these various components can be further organized and stored in a compact file format as described in a related U.S. Patent Application Ser. No. ______ (Attorney Docket No. PHI 3170).

The system and method embodiments described above may include a number of additional features. For example, the system may include a compressor/decompressor repository 492 that stores the actual algorithms for each of the compression and decompression techniques that are to be used along with definitions for their usage in schema instructions. In one embodiment, all or a portion of these algorithms may be stored in encrypted form in repository 492. Also, in 494, the encrypted algorithms may be stored in association with digital signatures that validate the encryptions. The digital signatures may or may not be stored with digital certificates approving of the usage of the schemas in the system.

Also, in some cases one or more blocks of comment/blank lines, or rows not covered by the regions defined in the compression schema, may be extracted and aggregated into a block, with their line numbers in the original text recorded. In this case, a predetermined type of text compression may then be applied, with the compressed block stored as an independent file part.

Data Decompression and File Reconstruction

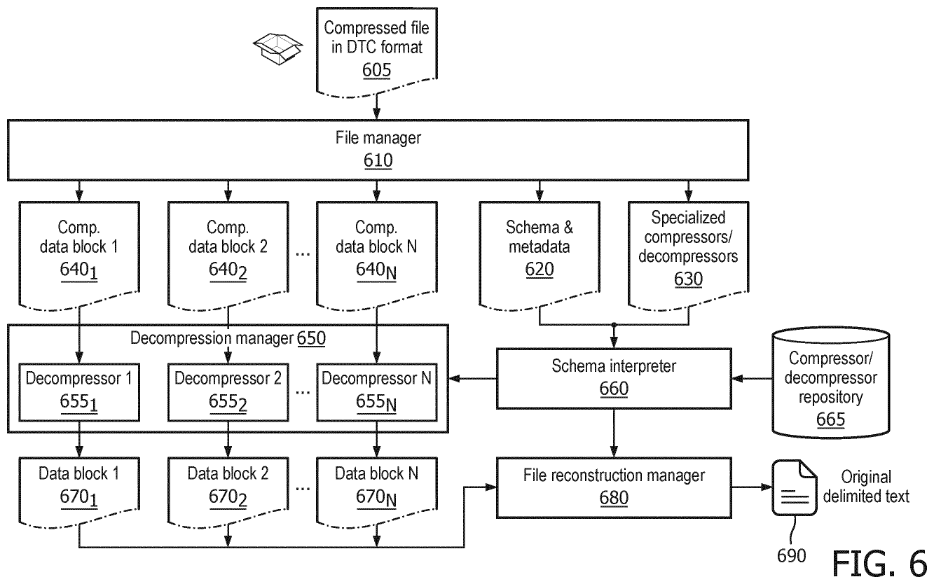

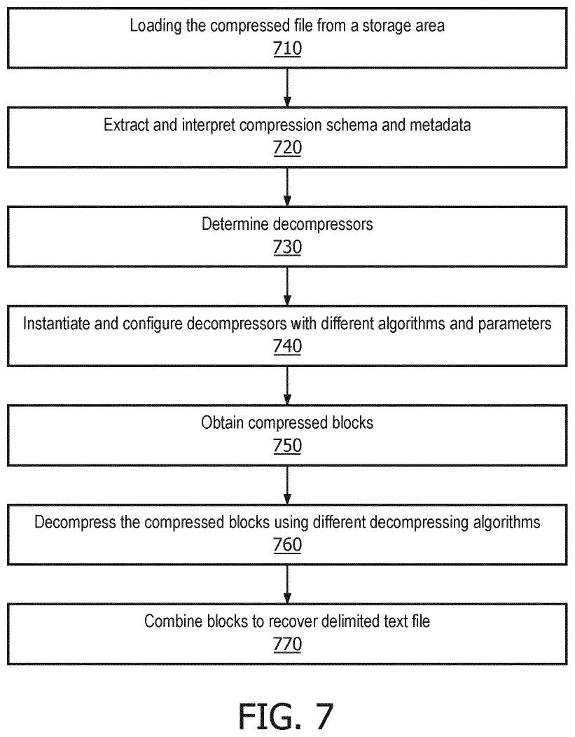

FIG. 6 illustrates an embodiment of a system for decompressing the compressed parts of the delimited text file and then reconstructing the decompressed parts to the delimited text file. FIG. 7 illustrates an embodiment of a method for performing the decompression and file reconstruction operations, which, for example, may be implemented using the system of FIG. 6.

Referring to FIGS. 6 and 7, the method includes, at 710, retrieving and loading the compressed file (e.g., in DTC format) 605 into the file manager 610 of the system. The file manager 610 may be the same file manager used during compression or a different file manager. The compressed file may be retrieved from a storage area, which, as previously indicated, may be an archive or another type of storage area. The compressed file may be retrieved, for example, in response to a request from an application or system that will use the compressed data (e.g., genomic data) for a research or other purpose. The request may be received from a local processor included in or connected to the processor or from a network. In this latter case, the archive or storage area may be, for example, a server, cloud storage, or other repository connected to the file manager through a network.

At 720, information 620 corresponding to the compression schema and metadata is extracted from the compressed file (or retrieved from a table stored for the compressed file) by the file manager. This information may itself be compressed using a predetermined compression algorithm known to the file manager. When the information corresponding to the compression schema and metadata are stored in encrypted and compressed form, the file manager may decrypt and decompress the compression schema information and metadata using a decompressor that reverses the compression performed by the known compression algorithm. As previously indicated, in one embodiment, the compression schema information and metadata may indicate, for example, not only the compression instructions (including the algorithms) for compressing the blocks of the delimited text file, but in some cases may also indicate one or more delimiter symbols used for the blocks and/or indexing information.

At 730, information on the decompression algorithms to be applied on different data blocks is extracted from the compression schema of the file. Based on the information, the codes of the decompression algorithms are then retrieved (e.g., verified, decrypted and/or decompressed) from the compressor/decompressor repository in 665 and/or the embedded modules of specialized compressors/decompressors in 630.

At 740, the decompression manager creates instances of (instantiates) a plurality of decompressors 6551 to 655N by loading the codes of their respective algorithms, setting any decompression parameters and allocating resources for computation and runtime storage for purposes of recovering the parts of the original delimited data file. While the number of decompressors is illustrated to be the same as the number of compressed blocks, this may not be the case in some embodiments. For example, each of the decompressors may decompress two or more of the compressed blocks, when the two or more blocks are compressed by the same algorithm.

The decompression manager 650 coordinates the decompressor instances to decompress the blocks using different corresponding algorithms based on information received by the schema interpreter 660, which may or may not be the same schema interpreter using during the decompression stage of the method. The schema interpreter reads and executes the instructions for decompression based on the schema information and metadata, and retrieves the codes of the decompression algorithms to be applied on the compressed data blocks. It then passes corresponding information to the decompression manager, which then decompresses the compressed blocks according to the directives from the schema interpreter. For example, decompression of each file part may be performed by one of the decompressors (compatible with the compression algorithm used) that has been instantiated based on the algorithm and parameters specified in the compression schema. To speed up the decompression process, decompression of the individual file parts or even individual data units may be performed in parallel.

In one embodiment, once the specific decompression algorithms and their corresponding parameters have been determined from the compression schema obtained through the file manager, the schema interpreter may retrieve the codes corresponding to the appropriate decompression algorithms from a repository 665 or embedded modules 630, and passes the codes and related parameters to the decompressor manager for instantiating the decompressors.

At 750, the compressed blocks 6401 to 640N are extracted from the bundled file by the file manager. As previously noted, N may be greater than or equal to one and the blocks may be compressed based on different compression algorithms.

At 760, the compressed blocks are input into the decompression manager 650. Once the decompressors have been instantiated and configured with the codes from the compressor/decompressor repository and/or embedded modules, the decompressors 6551 to 655N decompress the compressed blocks to recover the blocks of the delimited text file in their uncompressed form. The blocks may be stored, for example, in respective buffers for use by file reconstruction logic.

At 770, the file reconstruction manager 680 combines the now-uncompressed blocks 6701 to 670N to form the now-reconstructed original delimited text file 690. The file reconstruction manager may determine how to combine the uncompressed block in order to recover the reconstructed delimited text file based on the compression schema, metadata, and other information determined by the schema interpreter. This includes recombining lines, columns, blocks, or other portions of the blocks to reconstruct the original format of the delimited text file as it existed prior to deconstruction and compression. In one embodiment, reconstruction of the original file may be performed on a line-by-line basis, by extracting data elements from the buffers and assembling them with the insertion of the right delimiter symbols according to the compression schema and metadata.

The selective compression and decompression performed by the embodiments described herein may allow one or more blocks in one portion of the compressed delimited text file to be retrieved, decompressed, and reconstructed without retrieving, decompressing, and reconstructing blocks in other portions of the file. For example, a specific region (e.g., a specific range of one or more rows and/or one or more columns) containing information of interest to a user may be retrieved from the compressed data without retrieving and/or decompressing other portions of the compressed delimited text file. Thus, only the data of a multi-part delimited file may be retrieved and used that is of interest, in a manner that is independent from other parts of the file. This allows only targeted portions of a delimited text file to be selectively decompressed and accessed, which is beneficial for supporting fast query and random access.

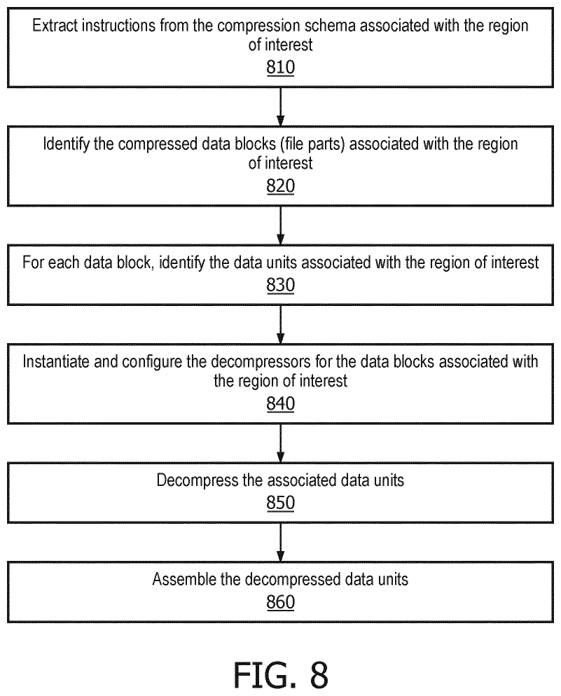

FIG. 8 illustrates an embodiment of a method that selectively accesses one or more blocks in the compressed delimited text file independent from accessing (e.g., decompressing, deconstructing, etc.) other portions of the file.

Referring to FIG. 8, the method includes, at 810, receiving information indicative of one or more regions of the compressed delimited text file that are of interest. The one or more regions of interest may correspond, for example, to a certain portion of a genomic data file. The information may be received, for example, by extracting instructions from the compression schema associated with the region of interest. In one embodiment, the region information may include a table/block identifier (ID), as defined in the compression schema, which identifies the portion(s) of the compressed delimited text file that is of interest.

At 820, the compressed data blocks (e.g., file parts) associated with the region(s) of interest are identified based on the instructions extracted from the compression schema. This operation may be performed, for example, by the schema interpreter.

At 830, for each data block identified in operation 820, one or more data units associated with the region(s) of interest may be identified.

For operations 820 or 830, or both, the part(s) (e.g., data blocks, data units) of the compressed delimited text file may be located, for example, in accordance with location information stored in a table accessed by the file manager. This may be accomplished, for example, in the following manner. First, the starting line number and the ending line number of the file part(s) of interest are mapped to corresponding block indices and offset line numbers in a block. This may be accomplished, for example, based on Equations (1) and (2).

Data_Unit_Index=Floor((Line_Number−Data_Block_Loc)/Data_Unit_Size)+1 (1)

Data_Unit_Offset=Line_Number−(Data_Unit_Index−1)*Data_Unit_Size (2)

In these equations, Data_Block_Loc is the block location, e.g., the beginning line number of the block in the original text, and Data_Unit_Size is the number of lines per data unit. Both elements may be indicated by information included in the compression schema. In the wherein a Row_Index of the table is used instead of Line_Number, then Data_Block_Loc may instead be the index of the first row of the block in the table.

To perform a query based on column values, the columns involved in the query conditions may be decompressed. Alternatively, a query can be performed on the search tree generated based on the column values and stored as a metadata component associated with the column. Then, the line numbers of the matching rows may be computed and Equations (1) and (2) may be used to determine the corresponding data unit block(s) and offset(s).

For all involved blocks, the blocks indicated in operation 840 may be identified and the relevant rows within the block(s) may be extracted using the computed line offsets.

At 840, the data decompression manager instantiates and configures the decompressors using the algorithm(s) and parameters specified for the data blocks associated with the region(s) of interest. This may involve configuring one of the decompressors or otherwise selecting a decompressor that has already been configured with the corresponding decompression algorithm.

At 850, the data units in the data block(s) associated with the region(s) of interest are decompressed by corresponding ones of the decompressors.

At 860, once decompression has taken place, the decompressed block(s) are assembled in the selected region according to the format defined in the compression schema. In one embodiment, a user may designate (by information in a user input) the output format of the extracted data units, for example, by specifying a reconstruction schema that describes how the blocks should be organized with semantics similar to that of a compression schema. The decompressed block(s) of interest may then be output in assembled form, for example, on a display, all without decompressing the blocks that are not of interest in the compressed delimited text file. In one embodiment, the region of interest for which the decompressed block(s) of interest are displayed may correspond to specific section of data in entire genomic information, for example, corresponding to a particular subject or sample of interest.

In accordance with one embodiment, a compression schema may be customized for the processing of virtual contact files (VCFs) and BED files using the proposed CDTC framework. In the following examples, we illustrate how a compression schema can be defined for respective the VCF and BED file formats.

VCF File Example

TABLE 1
A Simple VCF File Example (from IGSR)

##fileformat=VCFv4.0
##fileDate=20090805
##source=myImputationProgramV3.1
##reference=1000GenomesPilot-NCBI36
##phasing=partial
##INFO=<ID=NS,Number=1,Type=Integer,Description=“Number of Samples With Data”>
##INFO=<ID=DP,Number=1,Type=Integer,Description=“Total Depth”>
##INFO=<ID=AF,Number=.,Type=Float,Description=“Allele Frequency”>
##INFO=<ID=AA,Number=1,Type=String,Description=“Ancestral Allele”>
##INFO=<ID=DB,Number=0,Type=Flag,Description=“dbSNP membership, build 129”>
##INFO=<ID=H2,Number=0,Type=Flag,Description=“HapMap2 membership”>
##FILTER=<ID=q10,Description=“Quality below 10”>
##FILTER=<ID=s50,Description=“Less than 50% of samples have data”>
##FORMAT=<ID=GT,Number=1,Type=String,Description=“Genotype”>
##FORMAT=<ID=GQ,Number=1,Type=Integer,Description=“Genotype Quality”>
##FORMAT=<ID=DP,Number=1,Type=Integer,Description=“Read Depth”>
##FORMAT=<ID=HQ,Number=2,Type=Integer,Description=“Haplotype Quality”>

| | | | | | |
|---|---|---|---|---|---|
| #CHROM | POS | ID | REF ALT | QUAL FILTER INFO | FORMAT |

| NA00001 | | NA00002 | | NA00003 | | | |
|---|---|---|---|---|---|---|---|
| 20 | 14370 | rs6054257 | G | A | 29 | PASS | NS=3;DP=14;AF=0.5;DB;H2 |
| GT:GQ:DP:HQ 0\|0:48:1:51,51 1\|0:48:8:51,51 | | | | | 1/1:43:5:.,. | | |
| 20 | 17330 | . | T | A | 3 | q10 | NS=3;DP=11;AF=0.017 |
| GT:GQ:DP:HQ 0\|0:49:3:58,50 0\|1:3:5:65,3 | | | | | 0/0:41:3 | | |
| 20 | 1110696 | rs6040355 | A | G,T | 67 | PASS | NS=2;DP=10;AF=0.333,0.667;AA=T;DB |
| GT:GQ:DP:HQ 1\|2:21:6:23,27 2\|1:2:0:18,2 | | | | | 2/2:35:4 | | |
| 20 | 1230237 | . | T | . | 47 | PASS | NS=3;DP=13;AA=T |

| GT:GQ:DP:HQ 0\|0:54:7:56,60 0\|0:48:4:51,51 | | | | | 0/0:61:2 | | |
|---|---|---|---|---|---|---|---|
| 20 | 1234567 | microsat1 | GTCT | G,GTACT 50 | PASS | NS=3;DP=9;AA=G | GT:GQ:DP |

| | | |
|---|---|---|
| 0/1:35:4 | 0/2:17:2 | 1/1:40:3 |

With reference to the VCF file example in Table 1, the following compression schema may be applied using the following code as a possible (but not necessarily optimal) approach.

```
Delimiters = ‘\t’
Comment_Symbol = ‘##’
<Table ID=‘VCF_Example’ First_Row_Col_Names=True>
  <Blocks Data_Unit_Size=5>
    <Block Region_Cols=1 Data_Type = ‘uint’ Comp_Alg=‘RLE’
Block_Name=‘Chromosome’> </Block>
    <Block Region_Cols=2 Data_Type = ‘uint’ Comp_Alg=‘Delta’
Block_Name=‘Position’> </Block>
    <Block Region_Cols=3 Data_Type = ‘string’ Comp_Alg=‘Auto’ Block_Name=‘ID’>
</Block>
    <Block Region_Cols=4 Data_Type = ‘char’ Comp_Alg=‘Enum’ Block_Name=‘Ref’>
</Block>
    <Block Region_Cols=5 Data_Type = ‘char’ Comp_Alg=‘Enum’ Block_Name=‘Alt’>
</Block>
    <Block Region_Cols=6 Data_Type = ‘uint(8)’ Comp_Alg=‘Auto’
Block_Name=‘Quality’> </Block>
    <Block Region_Cols=7 Data_Type = ‘string’ Comp_Alg=‘Enum’
Block_Name=‘Filter’> </Block>
    <Block Region_Cols=8 Data_Type = ‘string’ Comp_Alg=‘VCF_Info($Comments)’
Block_Name=‘Info’>
    <Block Region_Cols=9 Data_Type = ‘string’ Comp_Alg=‘Enum’
Block_Name=‘Format’>
    <Block Region_Cols=10:12 Data_Type = ‘string’
Comp_Alg=‘VCF_Sample($Format)’>
    </Blocks>
</Table>
```

Note that in this example, two specialized compression algorithms “VCF_Info” and “VCF_Sample” are designed to process the Info and sample data (NA00001, NA00002, NA00003). For the VCF_Info method, the input argument $Comments indicates that the information in the Comments block should be used for identifying all variant attributes. The corresponding attribute values in the Info column are then extracted and stored as matrices per attribute to be compressed separately. For the VCF_Sample method, the input argument $Format indicates that the attributes (GT, GQ, DP, HQ) in the Format column should be used for splitting and organizing the data elements into their respective matrices for more effective compression of individual attributes.

BED File Example

TABLE 2
A Simple BED File Example (from UCSC Genome Browser FAQ)

browser position chr7:127471196-127495720
browser hide all
track name=“ItemRGBDemo” description=“Item RGB demonstration” visibility=2 itemRgb=“On”
chr7 127471196 127472363 Pos1 0 + 127471196 127472363 255,0,0
chr7 127472363 127473530 Pos2 0 + 127472363 127473530 255,0,0
chr7 127473530 127474697 Pos3 0 + 127473530 127474697 255,0,0
chr7 127474697 127475864 Pos4 0 + 127474697 127475864 255,0,0
chr7 127475864 127477031 Neg1 0 − 127475864 127477031 0,0,255
chr7 127477031 127478198 Neg2 0 − 127477031 127478198 0,0,255
chr7 127478198 127479365 Neg3 0 − 127478198 127479365 0,0,255
chr7 127479365 127480532 Pos5 0 + 127479365 127480532 255,0,0
chr7 127480532 127481699 Neg4 0 − 127480532 127481699 0,0,255

With reference to the BED file example in Table 2, the following compression schema may be applied using the following code as a possible (but not necessarily optimal) approach.

```
  Delimiters = ‘\t’
  <Table ID=‘BED_Example’ Region_Lines=3: First_Row_Col_Names=False>
    <Blocks Data_Unit_Size=5>
      <Block Region_Cols=1 Data_Type = ‘string’ Comp_Alg=‘Enum +
  RLE’ Block_Name=‘Chromosome’> </Block>
      <Block Region_Cols=2 Data_Type = ‘uint’ Comp_Alg=‘Delta’
  Block_Name=‘Chr_Start’> </Block>
      <Block Region_Cols=3 Data_Type = ‘uint’ Comp_Alg=‘Delta’
  Block_Name=‘Chr_End’> </Block>
      <Block Region_Cols=4 Data_Type = ‘string’ Comp_Alg=‘Auto’
  Block_Name=‘Name’> </Block>
      <Block Region_Cols=5 Data_Type = ‘uint(10)’ Comp_Alg=’Delta’
  Block_Name=‘Score’> </Block>
      <Block Region_Cols=6 Data_Type = ‘char(1)’ Comp_Alg=‘Enum +
  RLE’ Block_Name=‘Strand’> </Block>
      <Block Region_Cols=7 Data_Type = ‘uint’ Comp_Alg=‘Delta’
  Block_Name=‘Thick_Start’> </Block>
      <Block Region_Cols=8 Data_Type = ‘uint’ Comp_Alg=‘Delta’
  Block_Name=‘Thick_End’>
      <Block Region_Cols=9 Data_Type = ‘fstring(‘%uint(8) , %uint(8),
  %uint(8)’)’ Comp_Alg=‘RLE’ Block_Name=‘Item_RGB’>
    </Blocks>
</Table>
```

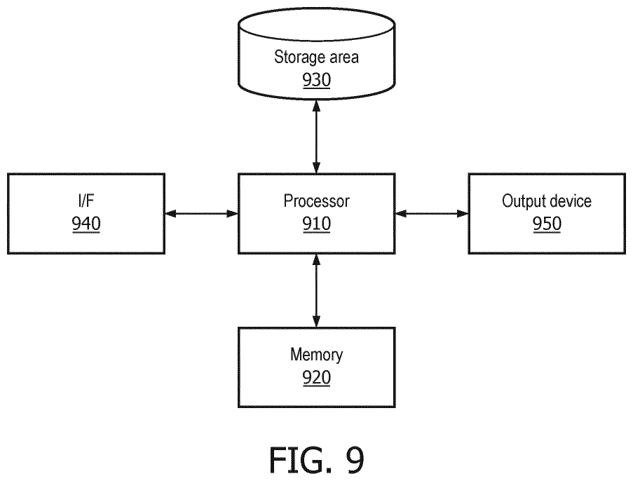

FIG. 9 illustrates an example of a processing system which may be used to perform the operations of the system and method embodiments described herein. The processing system includes at least one processor 910, a memory 920, a storage area 930, a communication interface 940, and an output device 950.

The at least one processor 910 may perform the operations of the managers, selectors, interpreters, parsers, and other information generating and processing operations described herein. In one embodiment, the processor 910 may have multiple cores, each dedicated to performing a different compression and/or decompression algorithm. In another embodiment, multiple processors may be included for performing different predetermined operations, including different compression/decompression algorithms and/or various other operations including parsing, schema generation, schema interpretation, and other operations associated with the embodiments. In one embodiment, the same processor may perform all of the compression and decompression. In so doing, the at least one processor 910 may perform the file construction and deconstruction operations and may generate the tables, data structures, and schemas, as well as interpret the schemas and perform generating and editing operations that allow a user to generate customized schemas.

The memory 920 may store instructions for causing the at least processor 910 to perform the operations of the system and method embodiments. The memory may be any one or combination of non-transitory computer-readable medium(s) locally connected to the at least one processor. In one embodiment, the processor and memory may be located in workstation used at a research facility, a laboratory, or other location where the information from the delimited text file may be used in connection with one or more intended applications. This is especially the case in the context of a delimited text file that stores genomic data.

The storage area 930 may be a database, repository, archive, or other storage area for storing the delimited text file, in original form, compressed form, or both. Like the memory, the storage area may be any one or combination of non-transitory computer-readable medium(s) locally connected to the at least one processor. In one embodiment, the storage area may be remotely connected to the at least one processor through a network connection. Such may be the case when, for example, the storage area 930 is included in a storage area network, cloud-computing network, or other processing and/or data storage architecture.

The communications interface (I/F) 940 may receive raw data, which may then be processed by the at least one processor 910 for forming the delimited text file. The processing may include converting the data into the text file format, with delimiters and other symbols and information described in connection with the compression schema discussed herein. The interface 940 may also receive requests issued in connection with the embodiments, as well as requests from other entities that may also have an interest in viewing or using the delimited text files.

The output device 950 may be a display which generates all or selected portions of the delimited text file stored and/or processed as described herein. This is especially useful when only a region of interest is to be output for analysis, in which case only block(s) of interest of a compressed delimited text file stored in the storage area 930 are decompressed for output, while other blocks not associated with the region of interest in the same file are not decompressed.

The methods, processes, and/or operations described herein may be performed by code or instructions to be executed by a computer, processor, controller, or other signal processing device. The code or instructions may be stored in a non-transitory computer-readable medium in accordance with one or more embodiments. Because the algorithms that form the basis of the methods (or operations of the computer, processor, controller, or other signal processing device) are described in detail, the code or instructions for implementing the operations of the method embodiments may transform the computer, processor, controller, or other signal processing device into a special-purpose processor for performing the methods herein.

The processors, interpreters, generators, parsers, extractions, editors, compressors, decompressors, managers, reconstructors, deconstructors, selectors, and other information generating, processing, and calculating features of the embodiments disclosed herein may be implemented in logic which, for example, may include hardware, software, or both. When implemented at least partially in hardware, the processors, interpreters, generators, parsers, extractions, editors, compressors, decompressors, managers, reconstructors, deconstructors, selectors, and other information generating, processing, and calculating features may be, for example, any one of a variety of integrated circuits including but not limited to an application-specific integrated circuit, a field-programmable gate array, a combination of logic gates, a system-on-chip, a microprocessor, or another type of processing or control circuit.

When implemented in at least partially in software, the processors, interpreters, generators, parsers, extractions, editors, compressors, decompressors, managers, reconstructors, deconstructors, selectors, and other information generating, processing, and calculating features may include, for example, a memory or other storage device for storing code or instructions to be executed, for example, by a computer, processor, microprocessor, controller, or other signal processing device. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the methods herein.

It should be apparent from the foregoing description that various example embodiments of the invention may be implemented in hardware or firmware. Furthermore, various exemplary embodiments may be implemented as instructions stored on a machine-readable storage medium, which may be read and executed by at least one processor to perform the operations described in detail herein. A machine-readable storage medium may include any mechanism for storing information in a form readable by a machine, such as a personal or laptop computer, a server, or other computing device. Thus, a machine-readable storage medium may include read-only memory (ROM), random-access memory (RAM), magnetic disk storage media, optical storage media, flash-memory devices, and similar storage media.

It should be appreciated by those skilled in the art that any block diagrams herein represent conceptual views of illustrative circuitry embodying the principles of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A method for compressing data, comprising:
obtaining a compression schema customized to a format of a delimited text file;
parsing the delimited text file into a plurality of data blocks based on the compression schema;
splitting each of the data blocks into a plurality of data units based on the compression schema; and
compressing the plurality of data units in the plurality of data blocks using different compression algorithms, wherein the delimited text file is parsed into the plurality of data blocks based on the region definitions in the schema; each of the plurality of data blocks is split into the plurality of data units based on its respective data unit size in the schema; and the plurality of data units in each of the plurality of data blocks are compressed using the different compression algorithms indicated by the compression instructions in the schema.

2. The method of claim 1, wherein obtaining the compression schema includes:
creating a new compression schema or determining the best-matching one from a plurality of compression schemas based on information input by a user or the extension of the delimited text file, wherein each of the plurality of compression schemas is customized for respective one of a plurality of different formats of delimited text files.

3. The method of claim 1, wherein obtaining the compression schema includes:
automatically analyzing or detecting the format of the delimited text file; and
automatically generating a new compression schema for optimum compression performance or selecting the best-matching one from a plurality of compression schemas stored in a schema repository, wherein each of the plurality of compression schemas is customized for respective one of a plurality of different formats of delimited text files.

4. The method of claim 3, wherein files corresponding to the compression schemas stored in the schema repository have predetermined file extension indicative of the plurality of different formats of the delimited texts files.

5. The method of claim 1, further comprising:
creating the compression schema customized to the format of the delimited text file based on a tool with a graphical user interface, the graphical user interface including predetermined windows to allow for input of information that customizes the compression schema to the format of the delimited text file.

6. The method of claim 1, further comprising:
generating a compressed file consisting of the plurality of compressed data units in the plurality of data blocks, and a compression schema that includes instructions for decompression of the plurality of compressed data units and file reconstruction of the compressed file.

7. The method of claim 6, wherein the compressed file includes metadata information for decompression, file reconstruction, and extended functionalities.

8. The method of claim 7, wherein the extended functionalities include data security and search query.

9. The method of claim 6, wherein the compressed file includes code and usage definitions of specialized compression/decompression algorithms for portability and accessibility of the compressed file.

10. The method of claim 1, wherein the compression instructions indicate the different compression algorithms and their corresponding parameters to be used to compress different ones of the plurality of units based on different content of the blocks.

11. The method of claim 10, wherein compression instructions indicate:
a first type of compression algorithm is to be used to compress a first data unit including a first one of the group consisting of a type of values, a type of information, a type of data format, and a type of data arrangement, and
a second type of compression algorithm is to be used to compress a second data unit including a second one of the group consisting of a type of values, a type of information, a type of data format, and a type of data arrangement, wherein the first one of the group is different from the second one of the group.

12. The method of claim 2, wherein determining the compression schema includes:
determining the compression schema from a plurality of compression schemas,
wherein each of the plurality of compression schemas is customized to include decompression information for respective one of a plurality of different formats corresponding to the compressed file.

13. The method of claim 12, wherein determining the compression schema includes selecting the compression schema from the plurality of compression schemas stored in a schema repository.

14. A method for selective data access, comprising:
receiving information indicative of a region of interest in the data (e.g. range of rows and columns in a table), the region of interest corresponding to one or more data units included in at least one data block in the compressed file;
selectively decompressing the one or more data units of at least one data block associated with the region of interest in the compressed file without decompressing other data units in the at least one data block or other data blocks in the compressed file, the one or more data units selectively decompressed based on one or more decompression algorithms indicated by the compression instructions in the compression schema;
reconstructing the region of interest from the selectively decompressed one or more first data units, the region of interest reconstructed based on the region definitions in the compression schema or any user-defined output format; and
outputting information indicative of the reconstructed region of interest.

15. The method of claim 14, further comprising:
selectively accessing the one or more data units based on a query of the compressed file, the query performed based on one or more terms or range of values found in one or more data units that are selectively decompressed.

16. The method of claim 14, wherein the delimited text file includes genomic information and wherein the region of interest can correspond to a selected range of genomic coordinates or gene IDs.

17. A system for compressing data, comprising:
a schema manager configured to allow users to create, select or auto-generate a compression schema customized to a format of a delimited text file;
a parser configured to parse the delimited text file into a plurality of blocks based on the region definitions in the compression schema;
a splitter configured to split each of the blocks into a plurality of data units based on its respective data unit size specified in the compression schema; and
a compression manager configured to compress the plurality of data units in the plurality of data blocks using different compression algorithms indicated by the compression instructions in the compression schema.

18. The system of claim 17, wherein the schema manager is to create a new compression schema or determine the best-matching one from a plurality of compression schemas based on information input by a user or the extension of the delimited text file, wherein each of the plurality of compression schemas is customized for respective one of a plurality of different formats of delimited text files.

19. The system of claim 17, wherein the schema manager is to automatically analyze or detect the format of the delimited text file, and automatically generate a new compression schema for optimum compression performance or select the best-matching one from a plurality of compression schemas stored in a schema repository, wherein each of the plurality of compression schemas is customized for respective one of a plurality of different formats of delimited text files.

20. The system of claim 17, wherein the compression manager is to obtain the codes of the compression algorithms from the user or the compressor repository, instantiate the compressors for each data block by allocating computational resources and memory, and running and monitoring the compression of the data units.
